(19) United States
(12) Patent Application Publication
(10) Pub. No.: US 2007/0179495 A1
(43) Pub. Date: Aug. 2, 2007

(54) Combination electrosurgery

(76) Inventors: Mathew E. Mitchell, Pelham, NH (US); Emma Wright, Arlington, MA (US)

Correspondence Address: FISH & RICHARDSON P.C.;SMITH & NEPHEW, INC., 150 Minuteman Road, Andover, MA, 01810, US

(21) Appl. No.: 11/340,858

(22) Filed: Jan. 27, 2006

(51) Int. Cl. A61B 18/14; A61F 7/00; A61F 7/12

(52) U.S. Cl. 606/41; 607/99

(57) ABSTRACT

An electrosurgical apparatus includes a probe having a first electrode surface area for performing a first electrosurgical procedure and a second electrode surface area for performing a second electrosurgical procedure. The second electrosurgical procedure is different from the first electrosurgical procedure. The apparatus includes the second electrode surface area overlapping the first electrode surface area and/or a masking device operable to mask at least a portion of the first electrode surface area.

### TECHNICAL FIELD

This disclosure relates to combination electrosurgery.

### BACKGROUND

There are a variety of different electrosurgical procedures, each of which may be performed using a different probe. Two common arthroscopic electrosurgical procedures are ablation of soft tissue and debridement or smoothing of fibrillated cartilage, such as thermal chondroplasty.

The first procedure, ablation of soft tissue, often includes high power radio frequency (RF) energy delivery in an “ablative” mode in order to aggressively and rapidly remove unwanted tissue. The electrode surface area is typically large to increase the amount of tissue that can be ablated in a single pass and has raised edges in order to create high current densities for ablation. The resulting cell death from tissue ablation is tolerated because retention of tissue viability is typically not a requirement.

The second procedure, debridement of fibrillated cartilage, typically has a goal of smoothing fibrillated cartilage to restore surface topography while retaining as much viable cartilage as possible. Therefore, for debridement, while still delivering energy in an ablative mode, the probe electrode typically operates at a much lower power, to avoid damage to the underlying cartilage, and the electrode surface area is much less than with the high power ablation described above, allowing the probe to ablate at substantially lower power resulting in a precise and controlled ablation of the fibrillated surface.

### SUMMARY

During a procedure to treat a joint disorder, such as an arthroscopic procedure on a knee joint, surgeons often use a device such as a shaver blade or an RF energy probe. However, during the course of the procedure, surgeons can encounter secondary disorders, such as fibrillated cartilage within the knee joint. Surgeons often attempt to smooth out the fibrillated cartilage in this scenario with the readily available device already opened for treating the primary disorder. However, neither a shaver blade nor an RF energy ablation probe is specifically designed for treating articular cartilage. For example, mechanical debridement with a shaver blade typically does not fully restore a smooth surface topography and can result in loss of excess healthy tissue. In contrast, an RF energy ablation probe can smooth the tissue, but typically results in excessive underlying cell death as discussed above.

Particular embodiments provide a single probe for performing two or more procedures. For example, a probe is provided for performing both soft tissue ablation and smoothing of fibrillated cartilage. Such probes allow a surgeon to avoid the added cost of opening another probe, and the added time and inconvenience of connecting another probe, while performing an operation that includes two or more electrosurgical procedures.

According to a general aspect, an electrosurgical apparatus includes a probe having a first electrode surface area for performing a first electrosurgical procedure and a second electrode surface area for performing a second electrosurgical procedure. The second electrode surface area overlaps the first electrode surface area, and the second electrosurgical procedure is a different procedure from the first electrosurgical procedure.

Implementations of this aspect may include one or more of the following features.

The first electrosurgical procedure includes ablating tissue, shrinking tissue, and/or smoothing tissue.

The electrosurgical apparatus includes a switch for selectively activating one or more of the first electrode surface area and the second electrode surface area. The switch includes a masking device operable between at least two positions. A first switch position masks at least part of the first surface area and a second switch position masks at least part of the second surface area. The switch is positioned on a handle of the probe.

The electrosurgical apparatus includes a generator having a switch for selectively activating either the first electrode surface area or the second electrode surface area. The generator automatically selects a power level based on an indication of which electrode surface area is active and/or an impedance detected by the electrosurgical apparatus. The indication of which electrode surface area is active is based on a position of a switch for selecting the first or the second electrode surface area.

In another general aspect, an electrosurgical apparatus includes a probe having a first electrode surface area for performing a first electrosurgical procedure and a second electrode surface area for performing a second electrosurgical procedure. The second electrosurgical procedure is a different procedure from the first electrosurgical procedure. The probe includes a masking device operable to mask at least a portion of the first electrode surface area.

Implementations of this aspect may include one or more of the following features.

The first electrode surface area is electrically isolated from the second electrode surface area. The first electrode surface area is on an opposite side of the probe from the second electrode surface area. The first electrode surface area is on a common side of the probe with respect to the second electrode surface area. The masking device is operable between at least two positions. A first switch position masks the portion of the first electrode surface area and a second switch position masks a portion of the second electrode surface area. The first position and the second position are offset from each other circumferentially with respect to the probe.

In another general aspect, an electrosurgical apparatus includes a probe having a first electrode surface area for performing a first electrosurgical procedure and a second electrode surface area for performing a second electrosurgical procedure. The probe includes a switch which selects at least one of the first electrode surface area, the second electrode surface area, or a combination of the first electrode surface area and the second electrode surface area.

Implementations of this aspect may include one or more of the following features.

The switch includes a masking device operable between at least two positions. A first switch position masks at least part of the first surface area and a second switch position masks at least part of the second surface area. The switch is positioned on a handle of the probe.

The electrosurgical apparatus includes a generator. The generator automatically selects a power level based on an indication of which electrode surface area is active and/or an impedance detected by the electrosurgical apparatus.

In another general aspect, a method includes identifying in an operating environment one or more tissue areas for a first electrosurgical procedure and one or more tissue areas for a second electrosurgical procedure. The method includes selecting a first electrode surface area on a probe to be an active surface area, performing the first electrosurgical procedure using the selected first electrode surface area of the probe to modify the one or more tissue areas, selecting a second electrode surface area on the probe, by masking the first electrode surface area, to be the active surface area, and performing the second electrosurgical procedure using the selected second electrode surface area to modify the one or more tissue areas.

Implementations of this aspect may include one or more of the following features.

The method includes reselecting the first electrode surface area after selecting the second electrode surface area, and performing the first electrosurgical procedure again, after performing the second electrosurgical procedure, using the reselected first electrode surface area of the probe.

The first electrosurgical procedure includes at least one of the group consisting of ablating tissue, shrinking tissue, and smoothing tissue. The method includes supplying power to the probe from a power source, and detecting a system impedance at the power source and a power setting of the power source.

One or more of the foregoing implementations provide the benefit of performing two or more electrosurgical procedures with a single probe. An electrosurgical apparatus or method incorporating one or more of the foregoing implementations will perform two or more electrosurgical procedures with the benefit of parameter feedback from the generator or probe, such as power settings, electrode settings, operating environment (tissue or saline), and real-time feedback of parameters such as voltage, current, and/or impedance.

The details of one or more embodiments are set forth in the accompanying drawings and the description below. Other features will be apparent from the description and drawings, and from the claims.

### DESCRIPTION OF DRAWINGS

FIG. 1 is a perspective view of a system for performing an electrosurgical procedure.

FIG. 2 is a perspective view of an electrode portion of an electrosurgical probe having a first electrode surface area and a second electrode surface area.

FIG. 3 is a flowchart of a procedure for performing an electrosurgical procedure with a single probe.

FIG. 4A is a perspective view of a distal portion of another embodiment of an electrosurgical probe having a first electrode surface area, a second electrode surface area, and a masking device.

FIG. 4B is a perspective view of the distal portion of the electrosurgical probe of FIG. 4A with the first electrode surface area masked by the masking device.

FIG. 4C is a perspective view of the distal portion of the electrosurgical probe of FIG. 4A with the second electrode surface area masked by the masking device.

FIG. 5A is a side sectional view of a distal portion of another embodiment of an electrosurgical probe with a masking device positioned in a maximum electrode exposure position.

FIG. 5B is a side sectional view of the distal portion of the electrosurgical probe of FIG. 5A with the masking device positioned in a partial electrode exposure position.

FIG. 5C is a bottom sectional view of the distal portion of the electrosurgical probe of FIG. 5A with the masking device positioned in the partial electrode exposure position.

FIG. 6 is a state diagram showing a procedure for controlling the electrosurgical probe of FIGS. 5A-5C.

FIG. 7A is a bottom sectional view showing electrode exposure for another embodiment of an RF probe with a masking device positioned in a partial electrode exposure position.

FIG. 7B is a bottom sectional view showing electrode exposure for the RF probe of FIG. 7A with the masking device positioned in a full electrode exposure position.

### DETAILED DESCRIPTION

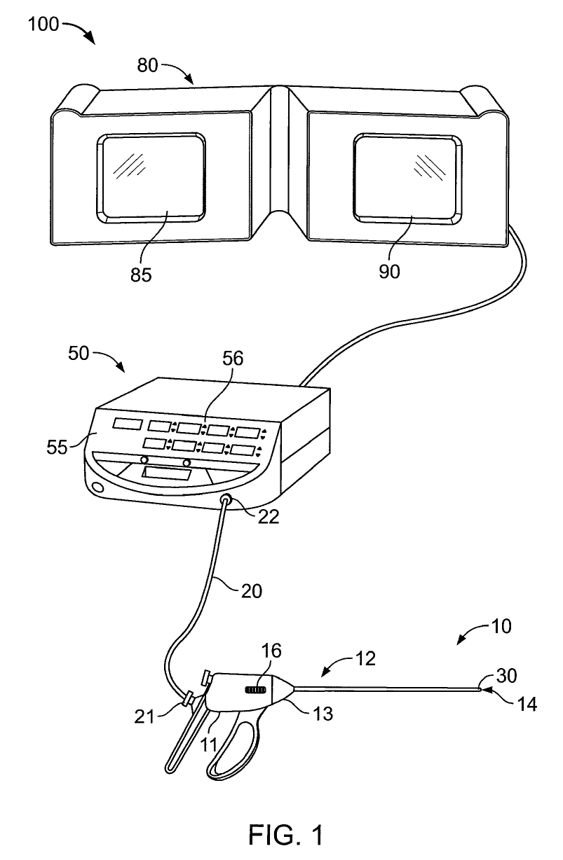

In FIG. 1, an electrosurgical system 100 includes an electrosurgical probe 0, a generator 50, a cable 20, and a pair of selection pedals 80. The single electrosurgical probe 10 can administer two or more different electrosurgical procedures for modifying tissue. The generator 50 delivers and/or controls a supply of energy, such as RF energy, to the electrosurgical probe 10 operating in a monopolar and/or bipolar mode. The selection pedals 80 permit a surgeon to select an electrosurgical procedure to be administered by the electrosurgical probe 10 and to select the appropriate power settings on the generator 50 for the selected procedure.

For example, the pair of selection pedals 80 shown in FIG. 1 include a CUT pedal 85 and a COAG pedal 90. When the surgeon or operator presses the CUT pedal 85, the electrosurgical probe 10 typically operates at a high power setting in an ablation mode. When the COAG pedal 90 is pressed, the electrosurgical probe 10 operates at a relatively lower power setting, such as in a thermal chondroplasty mode or coagulation mode.

The generator 50 includes a control unit 55 having one or more selection switches 56 for controlling power output of the generator 50. The power output of the generator 50 can be automatically controlled to a preset power setting. The preset power setting varies depending on which pedal 85, 90 is pressed. The power output of the generator 50 can also be manually controlled, such as by the selection switch 56 on the control unit 55 of the generator 50. The control unit 55 also monitors impedance and/or temperature feedback from the electrosurgical probe 10 and automatically adjusts and controls power levels delivered to the electrosurgical probe 10 in response to the impedance and/or temperature feedback.

The electrosurgical probe 10 includes a handle 11, and a shaft 12 extending from the handle 11. The shaft 12 includes a distal portion 14, and the probe 10 includes an electrode 30 operatively coupled to or integrally formed at the distal portion 14 of shaft 12 for applying energy to modify tissue (e.g., ablating, cutting, shrinking, or coagulating). The cable 20 operatively connects to the probe 10 and the generator 50 through a pair of cable plugs 21, 22, respectively. The probe 10 permits the surgeon to accomplish two or more different electrosurgical procedures with the same probe 10, such as soft-tissue, high-power ablation and treatment of thermal chondroplasty with the single probe 10. The probe 10 can include an optional control switch 16 permitting the operator to change between the operating modes or electrosurgical procedures that are administered with the probe 10.

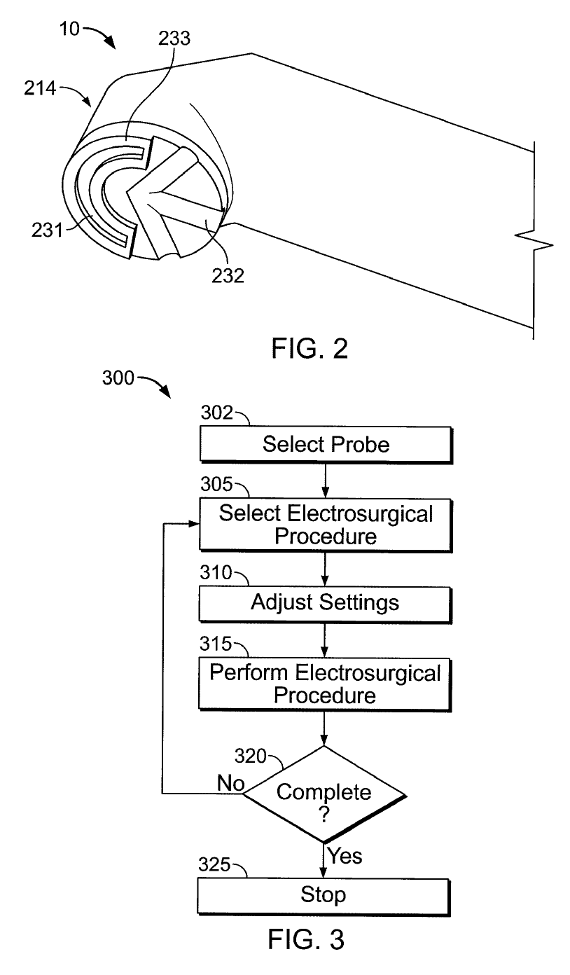

In FIG. 2, the probe 10 is provided with a distal portion 214 having a first electrode surface area 231 and a second electrode surface area 232. The first electrode surface area 231 and the second electrode surface area 232 act as separate, independently powered electrodes. The first electrode surface area 231 is sized and shaped to administer a first electrosurgical procedure, e.g., a smooth, arcuate band extending circumferentially around a portion of the first electrode surface area 231 for performing thermal chondroplasty, and the second electrode surface area 232 is sized and shaped, e.g., a multiple-pronged, arrow-shaped electrode band providing a relatively larger surface area with sharper edges to administer a different electrosurgical procedure such as soft-tissue, high-power ablation. The first and the second electrode surface areas 231, 232 are electrically insulated from each other with an intervening insulator 233 provided between the electrode surface areas 231, 232. A switch, such as the control switch 16 shown in FIG. 1, can be used to select either the first electrode surface area 231 or the second electrode surface area 232 as an active electrode surface area.

In FIG. 3, a process 300 is shown for using a probe that can administer two or more electrosurgical procedures. A surgeon selects an electrosurgical probe (302) according to a desired electrosurgical procedure, and the surgeon further selects an electrosurgical procedure to be administered on a target tissue (305) with one or more of the pedals 85, 90, control switch 16, and the control unit 55 of the generator 50. One or more settings are adjusted (310) manually and/or automatically by the manipulation of one or more of the control switch 16, the pedals 80, and the control unit 55 of the generator 50. The surgeon performs the electrosurgical procedure on the targeted tissue (315).

The surgeon determines whether all of the electrosurgical procedures are complete (320) by examining the target tissue area. If all of the electrosurgical procedures are complete (“yes” branch out of operation 320), the process 300 is stopped (325). If all of the electrosurgical procedures are not complete (“no” branch out of operation 320), the process 300 returns to operation 305 to select another procedure (305) to be performed (315) by the previously selected probe (302). To perform a second electrosurgical procedure, one or more settings are adjusted (310), for example, to select a different electrode or power setting. The surgeon performs the second selected procedure (315) and process 300 continues again to operation 320 to determine if further procedures are needed.

A surgeon can determine the necessity of administering a second electrosurgical procedure before, during, or after the first electrosurgical procedure. For example, during the initial examination of the target tissue area before the first procedure is performed, a surgeon can determine if a second electrosurgical procedure will be necessary. The various procedures can be performed on the same tissue or on different areas of tissue. Assuming that multiple procedures need to be performed, the selection of the electrosurgical probe (302) can include the selection of a combination probe for administering the identified first and second electrosurgical procedures. Alternatively, if a second electrosurgical procedure has not been identified by the surgeon during probe selection (302), a surgeon can determine the most likely secondary procedure that the surgeon would perform. The surgeon can then select (302) a combination probe having a first electrode surface area for the primary procedure and having a second electrode surface area for the most likely secondary procedure to be administered by the surgeon.

Process 300 may be used, for example, to perform a given procedure in its entirety before switching to another procedure. Alternatively, process 300 allows a surgeon to administer multiple procedures incrementally by switching back and forth between the multiple electrosurgical procedures. Two or more of the selection of the probe (302), the selection of the electrosurgical procedure (305), and the adjustment of settings (310) can be performed simultaneously or in orders different from the order shown in FIG. 3. For example, movement of the control switch 16 to a first position can select a first procedure (305) and automatically adjust settings (310) to a set of generator and probe settings corresponding to the first procedure, for example, generator and probe settings programmed into the control unit 55 or probe 10. Similarly, movement of the control switch 16 to a second position can select a second procedure (305) and adjust settings (310) to a different set of generator and probe settings corresponding to the second procedure, for example, different generator and probe settings programmed into the control unit 55 or the probe 10.

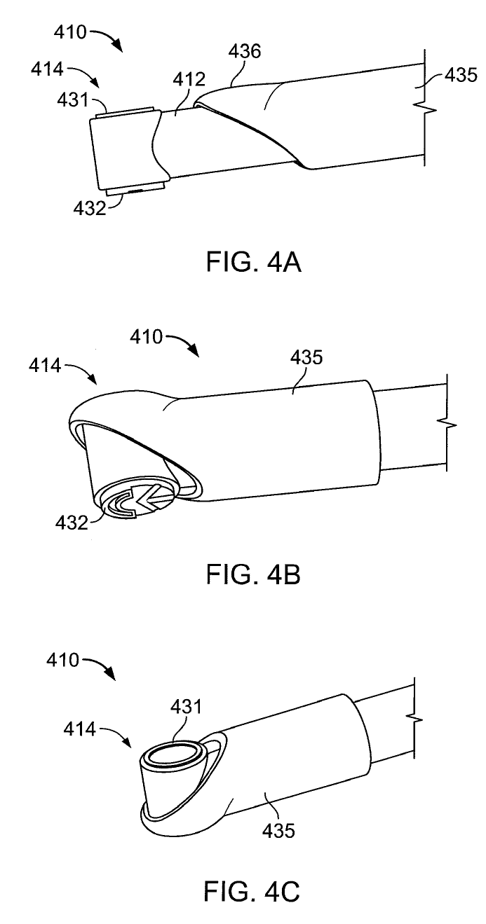

Other embodiments can include electrically coupled electrode surface areas. For example, in FIGS. 4A-4C, a probe 410 includes a distal portion 414 having a first electrode surface area 431 and a second electrode surface area 432. Rather than being separate, independently powered electrodes, the first electrode surface area 431 and the second electrode surface area 432 are electrically coupled to each other. The first and second electrode surface areas are provided on opposite sides of the distal portion 414. The probe 410 further includes a masking device 435, e.g., a retractable and/or rotatable insulating sheath, that permits an operator to select the effective electrode surface area that will be exposed for a particular electrosurgical procedure. The first electrode surface area 431 includes a circumferentially extending, relatively smooth and narrow electrode band extending around a circumference of the first electrode surface area 431 (FIG. 4C), e.g., for performing thermal chondroplasty. The second electrode surface area 432 includes a relatively larger surface area formed with sharper edges, e.g., a star-shaped electrode surface area for performing soft tissue, high power ablation.

The masking device 435 is an insulated sheath that is retractable and rotatable with respect to the first electrode surface area 431 and the second electrode surface area 432. For example, the masking device 435 can be formed with a distal portion 436 contoured to provide an interference- or snap-fit with the shaft 412 and electrode surface areas 431, 432. The surgeon alters the position of the masking device 435 by overcoming a relatively small locking force that permits the surgeon to reposition the distal portion 436 to cover the first electrode surface area 431 or the second electrode surface area 432. The distal portion 436 has a circular shape that corresponds to a relatively circular exterior of the first and second electrode surface areas 431, 432, so that the electrode is completely covered when the distal portion 436 covers one of the electrode surface areas, e.g., covers the first electrode surface area 431 (FIG. 4B).

Operation of the probe 410 can be described, for example, by referring to the process 300 of FIG. 3. Upon selecting an electrosurgical procedure (305) to administer, a surgeon can retract the sheath to a retracted position (as shown in FIG. 4A), rotate the sheath to selectively expose a first desired electrode surface area, and return the sheath to an extended position to mask the opposing electrode surface area (as shown in FIGS. 4B-4C). In certain embodiments, positioning of the masking device may be detected automatically by, for example, the generator 50 which may then, accordingly, select a corresponding procedure (305). The settings, e.g., of the generator 50 and the probe 410, for the selected procedure can be adjusted (310) manually, or automatically in response to, for example, detecting the positioning of the masking device 435. The surgeon performs the first electrosurgical procedure (315) on the targeted tissue. If all of the electrosurgical procedures are complete (“yes” branch out of operation 320), the process 300 is stopped (325). If all of the electrosurgical procedures are not complete (“no” branch out of operation 320), the process 300 returns to operation 305. The masking device 435 is then repositioned (305), thereby selecting another procedure (305) to perform with the probe 410.

In another embodiment of probe 410, the first and second electrode surface areas 431, 432 are electrically isolated rather than being electrically coupled to each other. A masking device or other switch can be used to select the surface area to receive power, or both surface areas (electrodes) may be powered simultaneously.

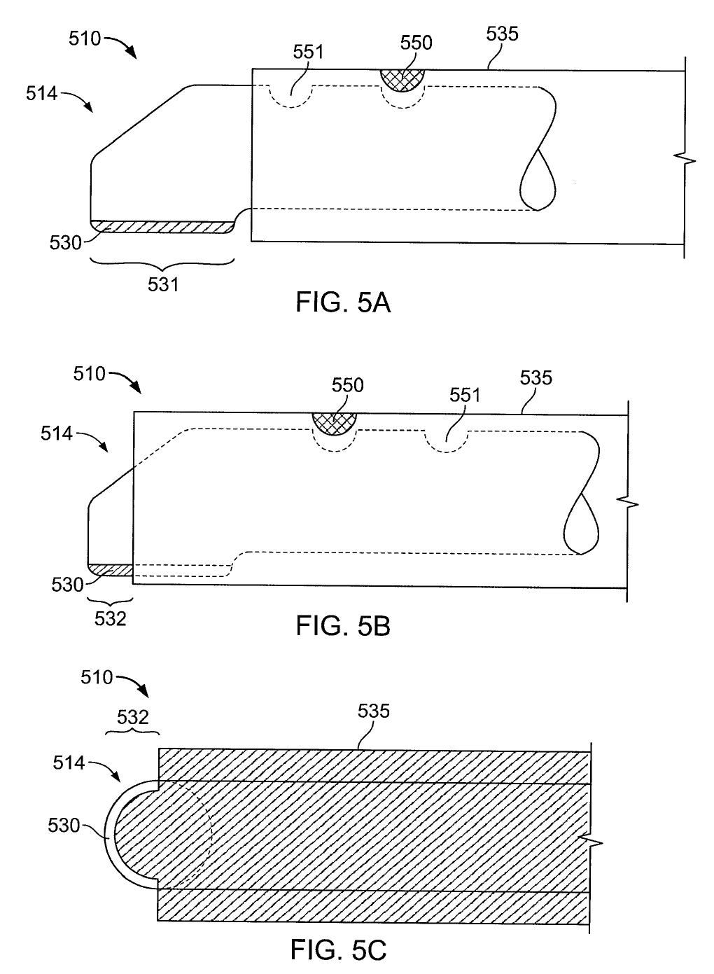

In FIGS. 5A-5C, another alternative probe 510 includes a distal portion 514 having a single electrode 530 and a masking device 535. The single electrode 530 includes a first electrode surface area 531 and a second electrode surface area 532 provided on the same side of the probe 510. The second electrode surface area 532 includes a relatively narrow, arcuate electrode band extending partially around a distal end of the electrode 530. The first electrode surface area 531 includes the remaining portion of the relatively circular shaped distal end of the electrode 530 and the second electrode surface area 532. Rather than being independently powered electrode surface areas, the first and the second electrode surface areas 531, 532 are electrically coupled to each other and therefore simultaneously powered whenever the probe 510 is powered. The masking device 535 is retractable and extendable between at least a first fully retracted position providing full exposure of electrode 530 (FIG. 5A), and at least one extended position providing partial exposure of electrode 530 (FIGS. 5B and 5C). The masking device 535 includes an optional, raised protuberance 550 formed along an interior surface of the masking device which engages with a pair of optional detents 551 formed in the exterior surface of the distal portion 514 o the probe 510. The raised protuberance 550 and the corresponding detents 551 provide the capability of indexing the masking device to predetermined positions and for maintaining the masking device in the predetermined positions. The clearance between the protuberance 550 and the detents 551 is sufficient to permit the surgeon to reposition the masking device 535 with a relatively small force. Alternatively, the protuberance 550 can be formed on the probe 510 and the detents 551 on the masking device 535. The masking device 535 can be spring biased to predetermined positions (not shown) and/or can include a raised groove and corresponding track configuration with indexed positions on either the masking device or probe, respectively.

In the fully retracted position for the masking device 535 shown in FIG. 5A, the first surface area 531 is exposed that includes, in this embodiment, the entire surface area of electrode 530. In the extended position for the masking device 535, shown in FIGS. 5B-5C, a second electrode surface area 532 is exposed that includes, in this embodiment, a relatively narrow distal portion of the electrode 530. The first and second electrode surface areas 531, 532 overlap, that is, share a common surface area. In this embodiment, the overlap consists of the entirety of the second electrode surface area 531.

Operation of the probe 510 can be described, for example, by referring to the process 300 of FIG. 3. Upon selecting an electrosurgical procedure (305) to administer, a surgeon can retract the masking device 535 to a retracted position (as shown in FIG. 5A) to selectively expose a desired electrode surface area, such as the first surface area 531 of the electrode 530. The settings, e.g., of the generator 50 and the probe 510, for the selected procedure can be adjusted (310) automatically in response to the positioning of the masking device 535. The surgeon performs the first electrosurgical procedure (315) on the targeted tissue. If all of the electrosurgical procedures are complete (“yes” branch out of operation 320), the process 300 is stopped (325). If all of the electrosurgical procedures are not complete (“no” branch out of operation 320), the process 300 returns to operation 305. The masking device 535 is then repositioned to select another procedure (305) with the probe 510.

The first and second electrode surface area 531, 532 can be designed for specific procedures, and power settings, for example, can be adjusted manually or automatically based on the position of the masking device 535. Alternatively, power settings can be the same regardless of the position of the masking device 535. In various embodiments, the masking device 535 can be extendable between the fully retracted position and a fully extended position, and can also, or alternatively, be operable between a multitude of alternative positions, e.g., indexed with position stops to numerous intermediate positions providing varying amounts of exposure between the fully retracted position and the fully extended position. In an embodiment, the fully extended position results in the entire surface area of electrode 530 being effectively masked and electrically insulated from any contact with surrounding tissue, permitting the probe to be effectively deactivated by the surgeon's positioning of the masking device 535.

The selection of an electrode surface area (and corresponding electrosurgical procedure) can optionally result in the initiation of probe and generator settings for the selected electrosurgical procedure, e.g., programmed into one or more of the control unit 55 or the probe 10, so that the surgeon does not have to manually adjust power settings on the control unit 55 of the generator 50. The generator can be provided with additional automated control features with one or more control algorithms designed to monitor, for example, temperature or impedance. For example, the generator can provide the ability to monitor impedance or temperature feedback from the electrosurgical probe and to automatically adjust and control power levels delivered to the electrosurgical probe in response thereto, e.g., to reduce the inappropriate administration of RF energy to a targeted tissue resulting in unnecessary cell death.

An exemplary control algorithm can be implemented that automatically monitors system parameters, such as the power level, impedance, percentage of electrode exposure, and/or operating environment, detected at, for example, the electrode to determine which electrode surface area has been selected by the surgeon. TABLE I includes test data for a probe as shown in FIGS. 5A-5C and having and electrode 530 for both high-power soft-tissue ablation and thermal chondroplasty. Eight operating states (1-8) for the probe 510 are shown in TABLE I that include recorded pedal 80 settings, average power settings, percentage of electrode exposure, the operating environment, and the average impedance.

TABLE 1
Impedance Measurements

| | Avg. Power | Electrode | | Avg. Impedance |
|---|---|---|---|---|
| State | Setting (W) | Exposure | Environment | (Ω) |
| 1 | 150 W | 100% | Saline | 110 |
| 2 | 150 W | 10% | Saline | 220 |
| 3 | 150 W | 100% | Tissue | 1500-2500 |
| 4 | 150 W | 10% | Tissue | >2500 |
| 5 | 60 W | 100% | Saline | 120 |
| 6 | 60 W | 10% | Saline | 180 |
| 7 | 60 W | 100% | Tissue | 160 |
| 8 | 60 W | 10% | Tissue | 1300-2000 |

An exemplary soft-tissue ablation procedure performed on a target tissue, e.g., articular cartilage from a knee joint operated on in an saline environment, typically requires a power setting of 150 W, electrode exposure of 100% (FIG. 5A), and an impedance between approximately 1500-2500 Ω (state 3). An exemplary thermal chondroplasty procedure performed on a target tissue typically requires a power setting of approximately 50-60 W (60 W shown in TABLE I), electrode exposure of 10% (FIGS. 5B-5C), and an impedance between approximately 1300-2000 Ω (state 8). If it is determined by the surgeon or system 100 that the probe is operating outside these ranges, the power to the probe 510 can be adjusted appropriately and operating parameters can be monitored for changes.

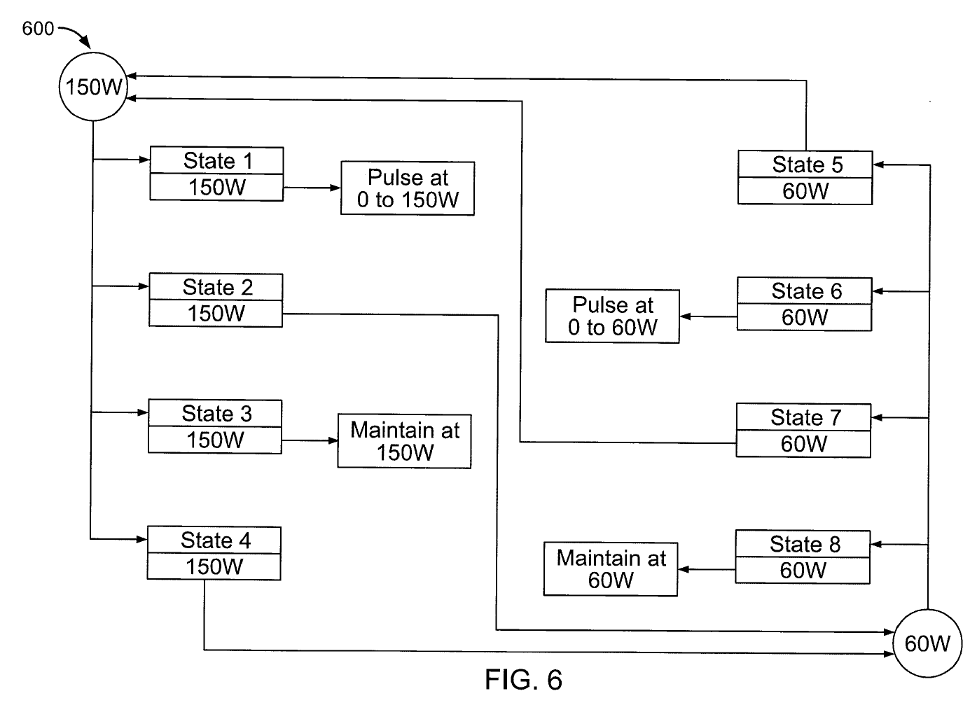

For example, FIG. 6 represents an exemplary control process 600 that can be implemented when the system 100 determines that a probe 510 is in a particular operating state. The system 100 can implement a “smart” probe 510 that automatically adjusts power settings based on detected operating conditions, such as impedance, electrode exposure, and whether the probe is engaging tissue or not (“saline” in TABLE I), and implements the control process 600 for the probe 510. In control process 600, if the power setting equals 150 W, the probe 510 is determined to be operating in any one of four 150 W operating states (1-4). Alternatively, if the power setting equals 60 W, the probe is determined to be operating in any one of four 60 W operating states (5-8). The control process 600 has two stable states (states 3 and 8) in which the control process 600 does not change the applied power. If other states are detected, control process 600 determines that the conditions are not desired, and adjusts accordingly as explained below.

In the first operating state (1), the system 100 detects a power setting of 150 W and an impedance of less than approximately 150 Ω (110 Ω shown in TABLE I). The control process 600 determines, e.g., based on previous empirical data, that the probe 510 is set at a 100% electrode exposure setting (FIG. 5A) and that the probe is not engaging tissue but is merely engaging the saline environment. Accordingly, the power settings are changed to a pulse power setting alternating between 0 and 150 W to prevent undesired tissue cell death arising from the application of continuous power while the probe is not touching tissue. The probe 510 can be provided with one or more sensors to permit periodic monitoring of operating parameters while in the pulse power setting so that power settings can be quickly returned to a constant setting, such as when the probe 510 engages tissue (and the measured impedance changes).

In the second operating state (2), the system 100 detects a power setting of 150 W and an impedance of between approximately 150 Ω and 500 Ω (220 Ω shown in TABLE I). The control process 600 determines that the probe 510 is set at a 10% electrode exposure setting and is operating in a saline environment. Because the electrode exposure of 10% is best suited for thermal chondroplasty of a tissue, the power setting is reduced to 60 W. The probe 510 is subsequently monitored to determine the new, applicable operating state (5-8) of the 60 W range.

In the third operating state (3), the system 100 detects a power setting of 150 W and an impedance of between approximately 1500-2500 Ω. The control process 600 determines that the probe 510 is set at a 100% electrode exposure setting and is operating in a tissue environment. Because these parameters are desirable for the soft-tissue ablation procedure, the power is maintained at 150 W.

In the fourth operating state (4), the system 100 detects a power setting of 150 W and an impedance of greater than 2500 Ω. The control process 600 determines that the probe 510 is set at a 10% electrode exposure setting and is operating in a tissue environment. Because the electrode exposure setting is more desirable for thermal chondroplasty, the power setting is reduced to 60 W. The probe 510 is subsequently monitored to determine the new, applicable operating state (5-8) of the 60 W range.

In the fifth operating state (5), the system 100 detects a power setting of 60 W and an impedance of less than or equal to approximately 170 Ω (120 Ω shown in TABLE I). The control process 600 determines that the probe 510 is set at a 100% electrode exposure. Because the electrode exposure setting is more desirable for soft-tissue ablation, the power setting is increased to 150 W. The probe 510 is subsequently monitored to determine the new, applicable operating state (1-4) of the 150 W range.

In the sixth operating state (6), the system 100 detects a power setting of 60 W and an impedance of greater than approximately 170 Ω and less than approximately 1000 Ω (180 Ω shown in TABLE I). The control process 600 determines that the probe 510 is set at a 10% electrode exposure and is operating in a saline environment. Accordingly, the power settings are changed to a pulse power setting alternating between 0 and 60 W to prevent undesired tissue cell death arising from the application of continuous power while the probe is not touching tissue. The probe 510 also permits periodic monitoring of operating parameters while in the pulse power setting so that power settings can be quickly returned to a constant setting, such as when the probe 510 engages tissue and the measured impedance changes.

In the seventh operating state (7), the system 100 detects a power setting of 60 W and an impedance of less than or equal to approximately 170 Ω (160 Ω shown in TABLE I). As with state 5, the control process 600 determines that the probe 510 is set at a 100% electrode exposure. Because the electrode exposure setting is more desirable for soft-tissue ablation, the power setting is increased to 150 W. The probe 510 is subsequently monitored to determine the new, applicable operating state (1-4) of the 150 W range.

In the eighth operating state (8), the system 100 detects a power setting of 60 W and an impedance of between approximately 1300-2000 Ω. The control process 600 determines that the probe 510 is set at a 10% electrode exposure and is operating in a tissue environment. Because these parameters are desirable for thermal chondroplasty, the power is maintained at 60 W.

TABLE II includes test data for a probe 510 as shown in FIGS. 5A-5C and having an electrode 530 sized and shaped for thermal chondroplasty. More specifically, the probe 510 was operated at either 50 W, 60 W, or 150 W settings, and in either an exposed mode (as in FIG. 5A) or a covered mode (as in FIGS. 5B-5C). The probe was operated at 50 W Covered, 60 W Covered, 150 W Covered, 60 W Exposed, and 150 W Exposed and monitored for cell death depth, debridement depth (depth of tissue removal), total cell damage (sum of cell death depth and debridement death), impedance, current, and actual power consumed while operating on cartilage samples from a knee joint in a saline environment. As explained below, the results shown in TABLE II suggest that the probe 510 is best suited for thermal chondroplasty when the power settings are approximately 50-60 W and with only partial electrode exposure.

TABLE II
Total Cell Damage

| | Cell Death | Debridement | Total Cell | Impedance | Current | Power |
|---|---|---|---|---|---|---|
| Configuration | Depth [μm] | Depth [μm] | Damage [μm] | (Ω) | (mA) | (W) |
| 50 W Covered | 151 | 113 | 264 | 1885 | 75 | 9.6 |
| 60 W Covered | 141 | 69 | 210 | 2691 | 62 | 9.4 |
| 150 W Covered | 357 | 111 | 468 | 3049 | 110 | 23.8 |
| 60 W Exposed | 1542 | 0 | 1542 | 135 | 719 | 50.2 |
| 150 W Exposed | 592 | 147 | 739 | 1637 | 198 | 45.6 |

In a typical thermal chondroplasty procedure, a surgeon desires to achieve debridement while avoiding unnecessary cell death. As seen in TABLE II, the minimum cell death is achieved while the probe 510 is operated at 50 W and 60 W with a partial electrode exposure. Further, “50 W Covered” and “60 W Covered” also achieve a desirable level of debridement.

TABLE II also reveals that debridement of the tissue is achieved at relatively low current values. In contrast, when the probe 510 is operated at 60 W in a fully exposed condition (FIG. 5A), nearly all of the relatively high current applied to the tissue resulted in high cell death depth without any debridement.

The system 100 can implement the control process 600 through an adjustment of, for example, the power settings of the generator 50 and the probe 10, and by detecting impedance with measurements taken at the generator 50 to obtain a system impedance, or across other system components to determine individual impedances, such as across the electrode 30 when operating in a bipolar mode. An impedance detection circuit within the generator will measure the system voltages and currents across the generator and/or other components, such as the electrode. A system impedance can be measured across the input and output of the generator, and component impedances can be derived by subtracting known impedances from the measured system impedance to determine component impedances or by direct measurements across the component. The generator 100 can be, for example, a VULCAN® generator sold by Smith & Nephew, Inc., of Memphis, Tenn. (catalog no. 7210812 or 7209673), the entirety of which is hereby incorporated by reference. The instructions for generator controls can be implemented in hardware or software, built into the generator 50 and/or the probe 10, or can be stored on one or more computer readable media, such as one or more memory cards or other portable memory media. The generator controls, particularly relating to electrosurgical power control, may include one or more of the features described in co-pending U.S. patent application Ser. No. 11/158,340, entitled Electrosurgical Power Control and filed on Jun. 22, 2005, the entirety of which is hereby incorporated by reference for all purposes.

Alternative control algorithms can be implemented that rely upon the interrelationships between various operating parameters, such as, for example, those shown in TABLES I and II. For example, as suggested in the discussion of TABLE II, other parameters, such as, for example, current can be used to automatically control settings for an electrosurgical procedure. Further, parameters other than those shown in TABLES I and II, such as, for example, current density (current per unit area of electrode exposure), can be used in a control algorithm.

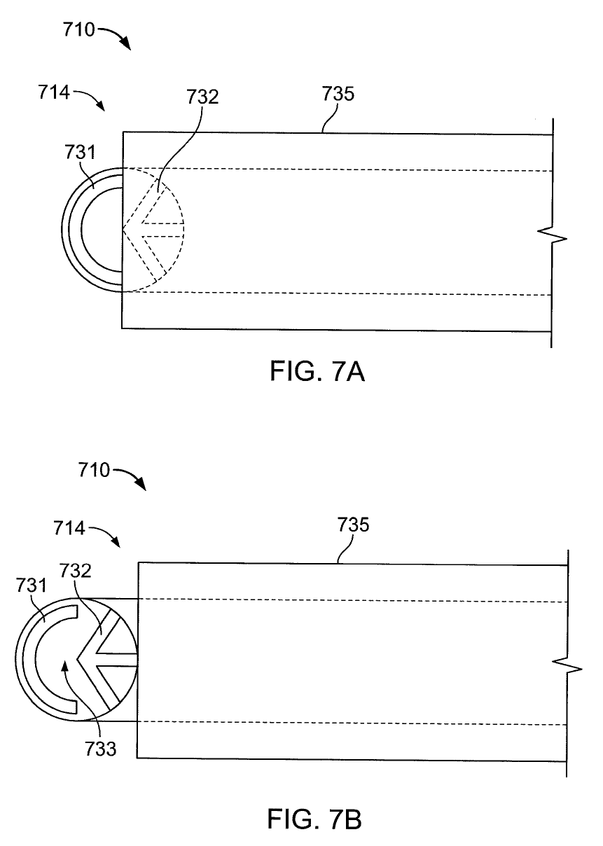

In FIGS. 7A-7B, a distal portion 714 of a probe 710 includes a first electrode surface area 731, a second electrode surface area 732, and a masking device 735. As described in connection with the probe 10 of FIG. 2, the first electrode surface area 731 and the second electrode surface area 732 act as separate, electrically isolated electrodes. However, rather than incorporating a switch 16 (FIG. 1) on a handle 11 (FIG. 1), the masking device 735 can be positioned to selectively control the activation of the first electrode surface area 731 or the second electrode surface area 732. When the masking device 735 exposes only the first electrode surface area 731, then the first electrode surface area 731 is powered on and the second electrode surface area 732 is powered off. When the masking device 735 exposes both first and second electrode surface areas 731, 732, only the second electrode surface area 732 is powered on. The first electrode surface area 731 is sized and shaped to administer a first electrosurgical procedure, e.g., thermal chondroplasty, and the second electrode surface area 732 is sized and shaped to administer a different electrosurgical procedure, e.g., soft-tissue, high-power ablation. The first electrode surface area 731 includes a relatively narrow, arcuate electrode band extending circumferentially around a perimeter of a distal end of the probe 710, e.g., for performing thermal chondroplasty. The second electrode surface area includes a multi-pronged, arrow-shaped electrode with relatively sharp edges, e.g., for performing soft tissue, high power ablation. The first and the second electrode surface areas 731, 732 are electrically insulated from each other with an intervening insulator 733 provided between the electrode surface areas 731, 732. The masking device 735 can also be fully extended to cover both electrode surface areas 731, 732 and to permit a surgeon thereby effectively to deactivate the probe 710.

Operation of the probe 710 can be described, for example, by referring to the process 300 of FIG. 3. Upon selecting an electrosurgical procedure (305) to administer, a surgeon can retract the masking device 735 to a retracted position (as shown in FIG. 7A) to selectively expose a desired electrode surface area, such as the first electrode surface area 731. The settings, e.g., of the generator 50 and the probe 510, for the selected procedure can be adjusted (310) automatically in response to the positioning of the masking device 735. The surgeon performs the first electrosurgical procedure (315) on the targeted tissue. If all of the electrosurgical procedures are complete (“yes” branch out of operation 320), the process 300 is stopped (325). If all of the electrosurgical procedures are not complete (“no” branch out of operation 320), the process 300 returns to operation 305. The masking device 735 is then repositioned to select another procedure (305) to perform with the probe 710.

Other embodiments of the probe 710 can power both the first and second electrode surface areas 731, 732 at the same time. When only one of the electrode surface areas 731, 732 is to be used for a procedure, a masking device can be positioned over the other surface area. Such a masking device can have one or more windows, for example, that can be positioned over one or more of the electrode surface areas 731, 732 to expose the surface area(s).

The probe 10 and the corresponding electrode 30 can be sized and shaped in a variety of configurations depending upon the targeted tissues and the desired electrosurgical procedures. For example, the probe 10 can be a monopolar probe (with a return electrode pad not shown) and/or a bipolar probe. Although a combination probe 10 has been described that can administer thermal chondroplasty or soft tissue ablation, alternative procedures utilizing monopolar and/or bipolar energy delivery modes can be accommodated with probes designed for specific electrosurgical procedures (and targeted tissues).

For example, the combination probe 10 can be a coagulation, an ablation, a shrinkage, and/or a smoothing probe. Ablation can be used as a therapeutic procedure or a non-therapeutic procedure. A non-therapeutic procedure may be, for example, using ablation to simply gain access to a target tissue area.

The combination probe can be a probe for performing one or more of the following tissue modification procedures, such as with an ablation probe at various power levels, including subacromial decompression, synovectomy, menisectomy, ACL/PCL debridement, meniscal debridement, labral resection, loose body excision, thermal chondroplasty, triangular fibrocartilage complex (TFCC) debridement, and scar tissue excision. The combination probe can be a probe for performing one or more of the following procedures, such as with a Ligament Chisel type probe configuration, including capsular release, lateral release, labral resection, capsular release, loose body excision, and TFCC debridement. The combination probe can be a probe for performing one or more of the following temperature controlled procedures, such as with a TAC™ probe, including capsulorrhaphy, chondroplasty, and medial plication. The combination probe can be a probe for performing one or more of the following procedures, such as with an ElectroBlade Resector probe, including subacromial decompression, synovectomy, CA ligament removal, and menisectomy.

Electrodes suitable for coagulation procedures can be an ablation-type electrode, e.g., provided with sharp edges, such as a SAPHYRE™ probe, or a shrinkage type electrode, e.g., provided with relatively smooth edges. Effective coagulation is dependent upon controlled power delivery, and therefore will typically require sub-ablative settings, including low voltage and high current to deliver the maximum heat to the targeted tissue. Suitable electrodes for tissue shrinkage typically have a smooth, contoured surface with no sharp edges. Power levels are typically sub-ablative and heat is relatively high to initiate tissue shrinkage.

The probe 10 can include a variety of options, including an electrode angled with respect to the shaft 12, e.g., 0-90 degrees, an electrode 30 having a relatively low or high profile, an electrode 30 with a suction feature to permit removal of modified tissue, and with temperature and/or impedance feedback. A combination probe 10 can utilize modifications of existing probes currently available for targeted electrosurgical procedures, such as the Ligament Chisel, EFLEX™, TAC™-S, ABLATOR™, GLIDER™, SAPHYRE™, and SCULPTOR™ probes available from Smith & Nephew, Inc., of Memphis, Tenn.

The combination probe can be directional, e.g., an ablation, shrinkage, or cartilage smoothing probe that is held by the surgeon in a specific orientation to administer a procedure. In contrast, the probe can be non-directional, that is, rotation of the probe around a longitudinal axis of the probe does not cause the probe to engage different tissue. For example, an angled electrode results in a directional probe, and a non-angled symmetrical electrode, e.g., a half dome, results in a non-directional probe.

A thermocouple (TC) can also be used in combination with shrinkage probes to monitor temperature and to adjust power settings while shrinking of the tissue progresses. Smoothing probes can have electrodes that are smooth, such as TAC™ (C II), that have sharp edges, and/or that have a relatively small surface area, such as GLIDER™. Smoothing with smooth electrodes is typically done in a sub-ablative mode and/or with temperature control. Smoothing with sharp electrodes is done in a controlled ablative mode, where electrode penetration is closely controlled and current output is minimized.

The control switch 16 can toggle electrode selection and initiate a routine that can include predetermined generator and/or probe settings. Alternatively, the probe can be provided with a probe recognition resistor in the handle 11 or shaft 12 that recognizes and identifies a selected electrode, e.g., such as an electrode surface area being selected with a masking device, and that sets the appropriate generator power for the selected electrode surface area. In lieu of a control switch positioned on the handle 11, the shaft 12, and/or the generator 50, the pedals 80 or the masking device can act as control switches for the probe 10. The pedals 80 or the masking device can automatically select the effective electrode surface area for a particular electrosurgical procedure and initiate generator and probe settings, for example settings programmed into one or more of the probe 10 or control unit 55.

The masking device, probe and insulator are preferably constructed of an insulating material, such as a material containing ceramic or plastic. The electrode is preferably constructed of a conductive material, such as a material containing tungsten or stainless steel. The masking device may be biased to return to an extended or retracted position, such as spring-biased to return the masking device of FIGS. 4A-4C to an extended position after the masking device has been rotated to the preferred side of the electrode. The masking device may alternatively, or in addition, be provided with incremental position stops to force the surgeon to overcome a minimum force before moving the masking device between position settings.

Although a combination probe 10 has been described in connection with two electrode surface areas, a probe can include three or more electrode surface areas, e.g., with a multiple position masking device rotatable through 180° (two electrode areas), 120° (three electrode areas), 90° (four electrode areas), etc.; and/or retractable through multiple extended positions creating any number of exposed, effective electrode surface areas. Alternatively, a multi-position switch can be used to select three or more independently powered, or electrically coupled, electrodes provided on the same probe.

A number of embodiments have been described. Nevertheless, it will be understood that various modifications may be made. For example, various device features and process steps from different embodiments may be combined, supplemented, modified, and/or deleted to form additional embodiments.

What is claimed is:

1. An electrosurgical apparatus comprising a probe, the probe including:
a first electrode surface area for performing a first electrosurgical procedure; and
a second electrode surface area for performing a second electrosurgical procedure, the second electrode surface area overlapping the first electrode surface area, and the second electrosurgical procedure being a different procedure from the first electrosurgical procedure.

2. The electrosurgical apparatus of claim 1, wherein the first electrosurgical procedure comprises at least one of the group consisting of ablating tissue, shrinking tissue, and smoothing tissue.

3. The electrosurgical apparatus of claim 1, further comprising a switch for selectively activating one or more of the first electrode surface area and the second electrode surface area.

4. The electrosurgical apparatus of claim 3, wherein the switch comprises a masking device operable between at least two positions, a first position that masks at least part of the first surface area and a second position that masks at least part of the second surface area.

5. The electrosurgical apparatus of claim 3, wherein the switch comprises a switch positioned on a handle of the probe.

6. The electrosurgical apparatus of claim 1, further comprising a generator.

7. The electrosurgical apparatus of claim 6, wherein the generator comprises a switch for selectively activating either the first electrode surface area or the second electrode surface area.

8. The electrosurgical apparatus of claim 6, wherein the generator is configured to automatically select a power level based on an indication of which electrode surface area is active.

9. The electrosurgical apparatus of claim 6, wherein the generator is configured to automatically select a power level based on an impedance detected by the electrosurgical apparatus.

10. The electrosurgical apparatus of claim 8, wherein the indication of which electrode surface area is active is based on a position of a switch for selecting the first or the second electrode surface area.

11. An electrosurgical apparatus comprising a probe, the probe including:
a first electrode surface area for performing a first electrosurgical procedure;
a second electrode surface area for performing a second electrosurgical procedure, the second electrosurgical procedure being a different procedure from the first electrosurgical procedure; and
a masking device operable to mask at least a portion of the first electrode surface area.

12. The electrosurgical apparatus of claim 11, wherein the first electrode surface area is electrically isolated from the second electrode surface area.

13. The electrosurgical apparatus of claim 11, wherein the first electrode surface area is on an opposite side of the probe from the second electrode surface area.

14. The electrosurgical apparatus of claim 11, wherein the first electrode surface area is on a common side of the probe with respect to the second electrode surface area.

15. The electrosurgical apparatus of claim 11, wherein the masking device is operable between at least two positions, a first position that masks the portion of the first electrode surface area and a second position that masks a portion of the second electrode surface area.

16. The electrosurgical apparatus of claim 15, wherein the first position and the second position are offset from each other circumferentially with respect to the probe.

17. An electrosurgical apparatus comprising a probe, the probe including:
a first electrode surface area for performing a first electrosurgical procedure;
a second electrode surface area for performing a second electrosurgical procedure; and
a switch configured for selecting at least one of the first electrode surface area, the second electrode surface area, or a combination of the first electrode surface area and the second electrode surface area.

18. The electrosurgical apparatus of claim 17, wherein the switch comprises a masking device operable between at least two positions, a first position that masks at least part of the first surface area and a second position that masks at least part of the second surface area.

19. The electrosurgical apparatus of claim 17, wherein the switch comprises a switch positioned on a handle of the probe.

20. The electrosurgical apparatus of claim 17, further comprising a generator, wherein the generator is configured to automatically select a power level based on an indication of which electrode surface area is active.

21. The electrosurgical apparatus of claim 17, further comprising a generator, wherein the generator is configured to automatically select a power level based on an impedance detected by the electrosurgical apparatus.

22. A method comprising:
identifying in an operating environment one or more tissue areas for a first electrosurgical procedure;
identifying in the operating environment one or more tissue areas for a second electrosurgical procedure;
selecting a first electrode surface area on a probe to be an active surface area;
performing the first electrosurgical procedure using the selected first electrode surface area of the probe to modify the one or more tissue areas;
selecting a second electrode surface area on the probe, by masking the first electrode surface area, to be the active surface area; and
performing the second electrosurgical procedure using the selected second electrode surface area to modify the one or more tissue areas.

23. The method of claim 17, further comprising:
reselecting the first electrode surface area after selecting the second electrode surface area; and
performing the first electrosurgical procedure again, after performing the second electrosurgical procedure, and using the reselected first electrode surface area of the probe.

24. The method of claim 17, wherein the first electrosurgical procedure comprises at least one of the group consisting of ablating tissue, shrinking tissue, and smoothing tissue.

25. The method of claim 17, further comprising:
supplying power to the probe from a power source; and
detecting a system impedance at the power source and a power setting of the power source.
